(19) United States
(12) Patent Application Publication
(10) Pub. No.: US 2026/0029332 A1
(43) Pub. Date: Jan. 29, 2026

(54) CHEMICAL MEASUREMENT SYSTEM AND METHOD

(71) Applicant: PENTAIR WATER POOL AND SPA, INC., Cary, NC (US)

(72) Inventors: Charles Bulger, Falcon Heights, MN (US); Robert Crowder, Lino Lakes, MN (US); Sarah Farley, White Bear Lake, MN (US); Jonathon Slye, Fridley, MN (US); Montie Roland, Sanford, NC (US)

(21) Appl. No.: 18/994,732

(22) PCT Filed: Jul. 17, 2023
(86) PCT No.: PCT/US2023/070300

§ 371 Date: Jan. 15, 2025

Related U.S. Application Data
(60) Provisional application No. 63/368,533, filed on Jul. 15, 2022.

(51) Int. Cl. G01N 21/25; G01N 21/78; G01N 21/85; G01N 33/18

(52) U.S. Cl. CPC G01N 21/251; G01N 21/78; G01N 21/85; G01N 33/182; G01N 33/1886; G01N 2021/8557

(57) ABSTRACT

A system for analyzing the water quality of a water sample is provided. The system includes an inlet designed to deliver the water sample to a photometric analyzer, a chemical reagent system downstream of the inlet and upstream of the photometric analyzer, and a controller. The chemical reagent system is designed to inject one or more reagents into the water sample. The photometric analyzer comprises a vial designed to contain the water sample, at least one light source designed to emit light toward a first side of the vial, and at least one light detector designed to detect the emitted light on a second side of the vial. The controller is configured to adjust a dosage of the one or more reagents injected into the water sample, receive data from the at least one light detector, and analyze the water quality of the water sample based on the received data.

### CROSS-REFERENCE TO RELATED APPLICATIONS

This application claims priority under 35 U.S.C. § 119 to U.S. Provisional Patent Application No. 63/368,533, filed Jul. 15, 2022, entitled “CHEMICAL MEASUREMENT SYSTEM AND METHOD,” the entire contents of which are incorporated herein by reference.

### FIELD OF THE INVENTION

The disclosure relates to a system and method for measuring chemicals in a water matrix. More particularly, the disclosure relates to an automated colorimeter for swimming pools.

### BACKGROUND OF THE INVENTION

Water quality testing is an important aspect of maintaining the clarity, safety, odor, and taste of water in aquatic systems. As used throughout, aquatic systems can include at least, for example, swimming pools, spas, hot tubs, drinking systems, reservoirs, potable water systems, incoming domestic or commercial water sources, and/or outputs or components associated with an aquatic system or water treatment systems such as a water softener or filtration system, agricultural applications (e.g., chemical spraying systems), and the like.

The water quality of aquatic systems can be based on one or more detected water quality parameters that may be provided as a measurement/value of one or more of pH, temperature, oxidation-reduction potential (ORP), hardness, alkalinity, cyanuric acid, free chlorine, chloramine, turbidity, total dissolved solids (TDS), sodium chloride, manganese, lead, mercury, fluoride, iron, copper, sulfate, bacteria/virus levels, and the like. An owner or aquatic system operator may determine the water quality themselves. However, testing water quality can be confusing, time-consuming, and may lack accuracy.

One known approach to measuring water quality is to use a manual pool management kit, such as a pH test strip kit. However, manual pool management kits may be unreliable and can lead to inaccurate results due to user error. Further, manual pool management kits may not have the versatility to test water from multiple sources or test for multiple water quality parameters simultaneously.

Another known approach to measuring water quality is the use of electronic probes and devices such as a pH probe, an ORP probe, and/or an ion-selective probe. However, electronic probes and devices can be expensive and difficult to use. For example, electronic probes may require frequent calibration. Further, electronic probes and devices may be limited in the number of water quality parameters that can be tested. Therefore, a manual test kit may still be needed to supplement the probe(s). Thus, electronic probes may not be a cost-effective or reliable option to test water quality.

Moreover, accurate and consistent water quality analysis can help reduce water treatment costs. In particular, it can be difficult in agricultural applications to determine the correct dosage of pesticides or other crop treatments to apply to the crops without an accurate water quality analysis. For example, if the pH, hardness, or TDS readings are not accurate, the dosage of pesticides or crop treatments applied to the crops may be incorrect. Thus, accurate and consistent water quality analysis can help reduce the quantity of chemicals required for crop treatments in agriculturally based aquatic applications.

Therefore, there is a need in the market for an automated chemical measurement system capable of testing multiple water quality parameters that are indicative of water quality without requiring multiple testing systems.

### SUMMARY

In one embodiment, a system for continuously testing a sample of water comprises a housing having a photometric analyzer, wherein the photometric analyzer is provided in the form of a spectrometer or a colorimeter. The system further includes a reagent injection manifold having a plurality of valves that are designed to isolate one or more individual reagents from the sample water. A reagent bank is also provided and includes a plurality of reagents that are in communication with the injection manifold. The system also includes a recirculation pump that at least provides the sample water from the photometric analyzer to the reagent injection manifold. A first solenoid valve is designed to control the flow of the sample of water into the system and a second solenoid valve is designed to control the flow of the sample of water out of the system. The first and the second solenoid valves isolate the sample of water for mixing and analysis when each of the first and second solenoid valves are in a closed position.

In some aspects, the reagent bank includes a chemical mechanism designed to thermally stabilize the plurality of reagents. In some instances, the chemical mechanism is deoxygenation of the plurality of reagents and a solvent, and dehydration of the plurality of reagents and the solvent.

In some aspects, the reagent bank includes a mechanical mechanism or electrical mechanism designed to thermally stabilize the plurality of reagents via a temperature regulation and/or cooling or heat removal system. The mechanical mechanism or electrical mechanism is provided in the form of one or more of a heat sink, a flow of cooling water provided to the reagent bank, a fan or other air movement device, a Peltier cooling system, or a refrigeration cycle.

The valves are provided in the form of one or more check valves, septum valves, or rotary valves.

The recirculation pump is designed to provide a driving force that causes the sample of water to flow through the chemical measurement system. In some forms, there is a separate and/or dedicated sample delivery pump.

In some forms, the chemical measurement system includes a pH and an ORP probe. The ORP probe is designed to differentiate between a true zero free chlorine reading or a false zero free chlorine reading.

The chemical measurement system includes a spectrometer that collects an absorbance spectrum to ensure the reagents are performing as anticipated. The photometric analyzer performs a chemical measurement test on the sample of water using a first dosing at a first time period and a second dosing at a second time period. The photometric analyzer further provides information on reagent degradation by comparing the results of the first dosing and second dosing.

In one embodiment, an automated chemical measurement system for testing a sample of water from a swimming pool is provided. The automated chemical measurement system includes a conduit that provides a sample of swimming pool water to the automated chemical measurement system. A chemical reagent system injects one or more chemical reagents into the sample of swimming pool water. A colorimeter is downstream and in fluid communication with the chemical reagent system, the colorimeter designed to analyze the sample of swimming pool water.

In some aspects, the chemical reagent system includes a reagent cartridge bank having the one or more chemical reagents, at least one pump, at least one valve to selectively isolate the one or more chemical reagents from the sample of swimming pool water, and an injection manifold.

In some aspects, the automated chemical measurement system further includes a first valve positioned in the conduit upstream of the chemical reagent system for controlling a flow of the sample of swimming pool water into the chemical reagent system of the automated chemical measurement system, and a second valve positioned in the conduit downstream of the colorimeter designed for controlling the flow of the sample of swimming pool water out of the automated chemical measurement system.

In some aspects, the reagent cartridge bank comprises a chemical mechanism for thermally stabilizing the one or more chemical reagents provided in the form of deoxygenation of the one or more chemical reagents and a solvent, or dehydration of the one or more chemical reagents and the solvent.

In some aspects, the reagent cartridge bank comprises a mechanical mechanism or an electrical mechanism for thermally stabilizing the one or more chemical reagents provided in the form of one or more of a heat sink, a flow of cooling water provided to the reagent cartridge bank, a fan or other air movement device, a Peltier cooling system, or a refrigeration cycle.

In some aspects, the colorimeter further includes a pH or an ORP probe.

In further aspects, the ORP probe differentiates between a true zero free chlorine reading or a false zero free chlorine reading.

In some aspects, the colorimeter is provided in the form of a photodetector, spectrometer, or photometric analyzer.

In additional aspects, the colorimeter collects and analyzes an absorbance spectrum to ensure the one or more chemical reagents is functioning as anticipated.

In some aspects, the colorimeter is designed to run a chemical measurement test on the sample of swimming pool water by comparing a result of a first measurement taken at a first time period and a second measurement taken at a second time period.

In some aspects, the colorimeter provides information on reagent degradation by comparing the first measurement and the second measurement.

In another embodiment, a system for analyzing water quality of a water sample of an aquatic system is provided. The automated chemical measurement system includes an inlet designed to deliver the water sample and a chemical reagent system downstream of the inlet, wherein the chemical reagent system is designed to inject one or more reagents into the water sample. A colorimeter is downstream of the chemical reagent system and includes a vial designed to contain the water sample, at least one light source designed to emit light toward a first side of the vial, and at least one light detector designed to detect the emitted light on a second side of the vial. A controller adjusts a dosage of the one or more reagents injected into the water sample, receives data from the at least one light detector, and analyzes the water quality of the water sample based on the received data.

In some aspects, the data includes how much emitted light was detected by the at least one light detector.

In further aspects, the chemical reagent system further includes a manually actuated syringe designed to permit a user to manually inject a reagent into the water sample.

In some aspects, the system is located in a bypass loop of the aquatic system.

In some aspects, the aquatic system includes a sanitizer, a water chemistry regulator, a filter, and a heater, and wherein the controller controls at least one of the sanitizer, the water chemistry regulator, the filter, and the heater in response to the analyzed water quality.

In some aspects, the chemical reagent system further comprises a reagent cartridge bank designed to contain one or more chemical reagents, at least one pump downstream of the reagent cartridge bank, the at least one pump designed to provide a driving force to deliver the one or more chemical reagents to the water sample. At least one valve is downstream of the at least one pump, the at least one valve designed to control a flow of the one or more chemical reagents. An injection manifold is downstream of the at least one valve, wherein the injection manifold is designed to inject the one or more chemical reagents into the water sample.

In some aspects, the controller is configured to control a flow rate of the one or more chemical reagents by adjusting the at least one valve from a first closed position to a second open position.

In some aspects, the chemical reagent system further includes a complementary metal-oxide-semiconductor sensor.

In another embodiment, a method of analyzing one or more water quality parameters of a water sample in a closed loop aquatic system is provided. The method comprises the steps of delivering the water sample to a vial, providing a reagent test bank having a reagent cartridge that contains a chemical reagent, a pump designed to control a dosage of the chemical reagent, a valve designed to control a flow of the chemical reagent, and an injection manifold designed to inject the chemical reagent into the water sample. The method further includes the steps of adding the chemical reagent to the water sample, emitting a light from a light source toward the vial, detecting the emitted light with a light detector adjacent the vial, and determining the one or more water quality parameters based on the detected emitted light.

### DESCRIPTION OF THE DRAWINGS

Examples are described with reference to the following drawing figures. The same numbers are used throughout the figures to reference features and components.

FIG. 1 is a schematic block diagram of an exemplary aquatic system in a swimming pool setting having an automated chemical measurement system in accordance with the disclosure;

FIG. 2 is a schematic block diagram of an automated chemical measurement system designed to be used in an aquatic system;

FIG. 3A is a detailed schematic block diagram of one embodiment of a chemical reagent system included in the automated chemical measurement system of FIG. 2;

FIG. 3B illustrates a detailed schematic block diagram of another embodiment of a chemical reagent system included in the automated chemical measurement system of FIG. 2;

FIG. 4 is a front elevational view of an automated colorimeter designed to be used with the chemical measurement system of FIG. 2;

FIG. 5 is a schematic block diagram of another embodiment of an automated chemical measurement system;

FIG. 6 is a schematic block diagram of yet another embodiment of an automated chemical measurement system;

FIG. 7 is a schematic of yet another embodiment of an automated chemical measurement system;

FIG. 8 is a top isometric view of a test stand that may retain one or more components of the automated chemical measurement systems described herein, with portions omitted for clarity;

FIG. 9 is a partial front elevational view of the test stand of FIG. 8;

FIG. 10 is a side isometric view of a housing that retains one or more components of the automated chemical measurement systems described herein; and

FIG. 11 is a side isometric view of the housing of FIG. 10, with some components rendered transparently to illustrate various internal components disposed within the housing.

Before explaining the disclosed embodiments of the present disclosure in detail, it is to be understood that the invention is not limited in its application to the detail of the particular arrangements shown since the invention is capable of other embodiments. Exemplary embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from the embodiments of the invention. Thus, embodiments of the invention are not intended to be limited to embodiments shown but are to be accorded the widest scope consistent with the principles and features disclosed herein. The following detailed description is to be read with reference to the figures, which like elements in different figures, have reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof, as well as additional items. Unless specified or limited otherwise, the terms “connected,” “supported,” “controlled,” and “communicated” and variations thereof are used broadly and encompass both direct and indirect connections, supports, controls, and couplings Further, “connected” and “communicate” are not restricted to physical or mechanical connections or couplings.

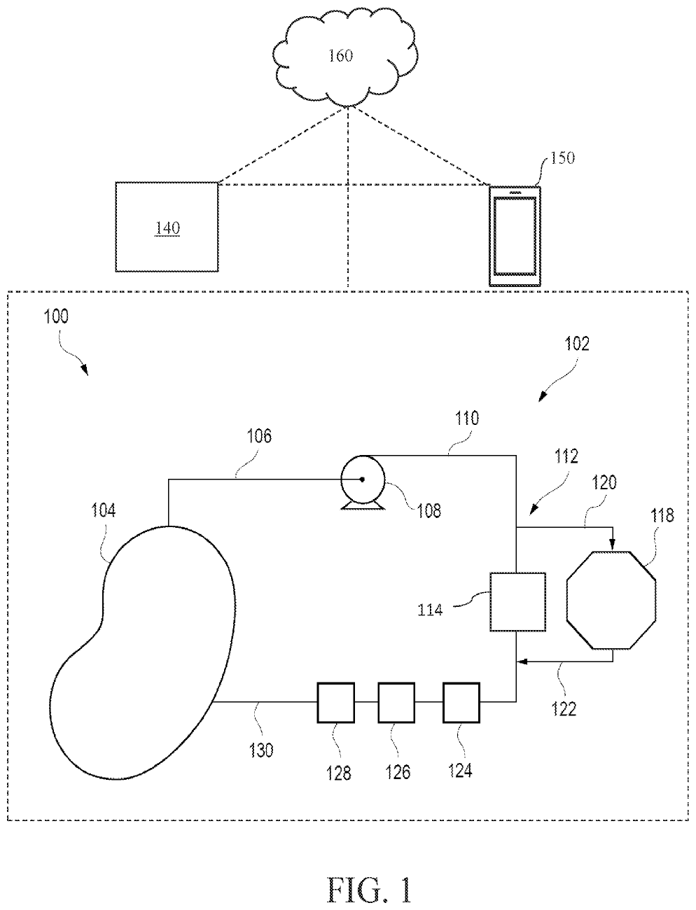

Turning to FIG. 1, a block diagram of an aquatic system 100 is depicted. The aquatic system 100 is provided in the form of one or more swimming pool components 102 designed for use with a swimming pool 104. The pool components 102 include plumbing (e.g., conduits) and one or more pool management devices that form a closed loop fluid (e.g., water) circuit. The pool components 102 include one or more of a pump inlet conduit 106, a pool pump 108 (e.g., variable speed drive), a pump outlet conduit 110, a bypass conduit 112, a pool filter 114, a sanitizer 124, a water chemistry regulator 126, a heater 128, and a discharge conduit 130.

Portions of water from the swimming pool 104 can flow from the swimming pool 104 through the pump inlet conduit 106 to a suction side of the pool pump 108. The pool pump 108 provides a driving force for the pool water to flow through the pump outlet conduit 110 to various other downstream components. The water from the swimming pool 104 also flows through the pool filter 114 and/or through the bypass conduit 112. After the water from the swimming pool 104 exits the pool filter 114 and/or the bypass conduit 112, the water from the swimming pool 104 can optionally be provided to or be in communication with other components in the aquatic system 100 (e.g., the sanitizer 124, the water chemistry regulator 126, and/or the heater 128) and return to the swimming pool 104 through the discharge conduit 130.

The bypass conduit 112 is designed to be in communication with an automated chemical measurement system 118, as described hereinbelow. A portion of the main water from the swimming pool 104 (i.e., a sample of the water) that is flowing through the pool plumbing (e.g., is disposed anywhere outside of the swimming pool 104 within the conduits or one or more of the pool components 102) can be provided to the automated chemical measurement system 118 through a branched take-off conduit 120. The take-off conduit 120 is designed to direct at least a portion of the water flowing through the pump outlet conduit 110 to enter the bypass conduit 112 and flow through the automated chemical measurement system 118. The take-off conduit 120 can be positioned downstream of the pool pump 108 and upstream of the pool filter 114. As discussed in greater detail below, the automated chemical measurement system 118 determines the pool water quality by analyzing one or more water quality parameters. The sample of the pool water can exit the automated chemical measurement system 118 through a return conduit 122. The return conduit 122 can rejoin the pool plumbing and tie into the discharge conduit 130 downstream of the pool filter 114 and upstream of the swimming pool 104.

A benefit of locating the automated chemical measurement system 118 in the bypass conduit 112 is that a continuous delivery of a fresh sample of water from the swimming pool 104 can be provided to the automated chemical measurement system 118 without the need for removing water from the swimming pool 104, the use of additional pumps, and without interrupting the operation and enjoyment of the swimming pool 104. The use of the pool pump 108 as the mechanism to provide the water sample to the automated chemical measurement system 118 can also reduce the amount of equipment needed in the aquatic system 100, thereby reducing equipment maintenance and cost.

Still referring to FIG. 1, the aquatic system 100 can include one or more of the sanitizer 124, the water chemistry regulator 126, and the heater 128 downstream of the bypass conduit 112. However, one or more components of the aquatic system 100 may be provided at different points in the fluid circuit or omitted. The sanitizer 124 and the water chemistry regulator 126 are designed to control one or more water treatment chemicals that are to be added to the swimming pool 104. In one embodiment, the sanitizer 124 is designed to add chlorine and/or bromine to the aquatic system 100. In one embodiment, the water chemistry regulator 126 is designed to add one or more of hydrochloric acid, sodium bisulfate, carbon dioxide, sulfuric acid, sodium carbonate, or other water treatment chemicals to the aquatic system 100. The heater 128 is optionally included and is designed to heat the water in the aquatic system 100.

The aquatic system 100 may further include a central controller 140 and a user device 150 that can interface with the central controller 140 either directly over a local area network or via a cloud network 160. The user device 150 can be provided in the form of a cell phone, tablet, or any other similar portable electronic device that includes a camera and a user interface.

Although FIG. 1 depicts the central controller 140 in communication with the user device 150 and the cloud network 160, it should be noted that various communication methodologies and connections may be implemented to work in conjunction with, or independent from, one or more local controllers associated with one or more individual components associated with the aquatic system 100 (e.g., a pump controller, a heater controller, a controller included in or associated with the automated chemical measurement system 118, etc.).

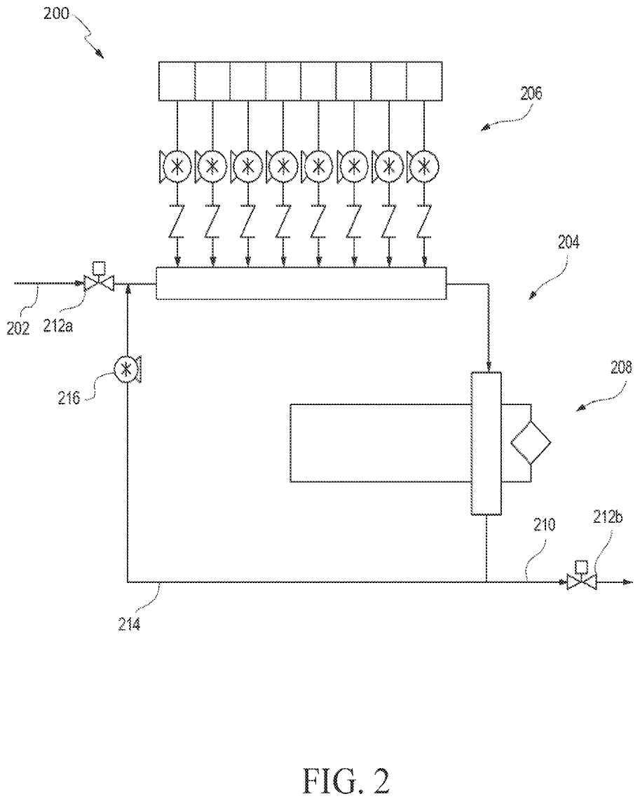

FIG. 2 illustrates a schematic block diagram of one embodiment of an automated chemical measurement system 200. In one embodiment, the automated chemical measurement system 200 is the automated chemical measurement system 118 of FIG. 1.

The automated chemical measurement system 200 can include a feed conduit 202, a branch conduit 204 in fluid communication with the feed conduit 202, and an outlet conduit 210 in fluid communication with the branch conduit 204. The feed conduit 202 can permit the water sample from the aquatic system 100 to flow into the branch conduit 204. In one embodiment, the feed conduit 202 is the take-off conduit 120 of FIG. 1. The feed conduit 202 can include a first solenoid valve 212a configured to control fluid flow through the feed conduit 202. Thus, the first solenoid valve 212a can control the water flow into the branch conduit 204.

The automated chemical measurement system 200 further includes a chemical reagent system 206, an automated colorimeter 208 downstream and in fluid communication with the chemical reagent system 206, a return conduit 214 downstream and in fluid communication with the automated colorimeter 208, and a secondary pump 216 positioned in the return conduit 214. In one embodiment, the branch conduit 204 is the bypass conduit 112 of FIG. 1. In some aspects, the secondary pump 216 may be omitted.

The chemical reagent system 206 can be positioned downstream of the first solenoid valve 212a and is designed to control a dosage rate of one or more chemical reagents injected into the water sample flowing through the branch conduit 204. The automated colorimeter 208 may be positioned downstream of the chemical reagent system 206 and configured to receive the water sample, including one or more injected chemical reagents. After the water sample passes through the chemical reagent system 206 and the automated colorimeter 208, the water sample can return to the feed conduit 202 via a return conduit 214 and/or exit the branch conduit 204 via the outlet conduit 210.

The return conduit 214 can tie into the feed conduit 202 downstream of the first solenoid valve 212a and upstream of the chemical reagent system 206. The return conduit 214 can include the secondary pump 216. The secondary pump 216 can be provided in the form of a recycle pump and/or a mixing pump that mixes the one or more reagents injected into the branch conduit 204 with the sample of water. Further, the secondary pump 216 can provide the driving force for the water sample to flow through the automated chemical measurement system 200.

The outlet conduit 210 can be in fluid communication with the aquatic system 100, such as the swimming pool 104 of FIG. 1. In one embodiment, the outlet conduit 210 is the return conduit 122 of FIG. 1. The outlet conduit 210 can include a second solenoid valve 212b positioned downstream of the automated colorimeter 208 that is designed to control the flow of the water sample out of the branch conduit 204.

Still referring to FIG. 2, during an automated water sample measurement operation, the first solenoid valve 212a is configured in an open position, permitting the water sample to flow into the branch conduit 204. The chemical reagent system 206 injects one or more chemical reagents into the water sample. The one or more chemical reagents mix with the water sample prior to the water sample entering the automated colorimeter 208. The one or more reagents injected into the branch conduit 204 may depend on the one or more water quality parameters being tested. As the mixed water sample passes through the automated colorimeter 208, the mixed water sample is analyzed to determine a value of one or more water quality parameters. The mixed water sample then exits the automated colorimeter 208. The mixed water sample can continue to circulate through the branch conduit 204 via the return conduit 214 and/or exit the branch conduit 204 via the outlet conduit 210 when the second solenoid valve 212b is in an open position.

During a mixing operation, the branch conduit 204 can be isolated when the first solenoid valve 212a and second solenoid valve 212b are configured in a closed position. In one embodiment, the secondary pump 216 can reverse the flow direction of the water sample to mix the water sample and the one or more reagents injected into the branch conduit 204.

During a rinsing operation, the first solenoid valve 212a and second solenoid valve 212b can be configured in an open position, which can allow for a continuous flow of water through at least a portion of the automated chemical measurement system 200 including the chemical reagent system 206 and the automated colorimeter 208. The rinsing mode can remove water samples that have been analyzed and deliver a fresh sample of water for additional analysis. In one embodiment, the secondary pump 216 can occasionally turn on and off while in the rinse mode (e.g., over a period of various seconds, minutes, or at other intervals). The secondary pump 216 pulsing can help ensure that a fresh sample of water is consistently present in each portion of the automated chemical measurement system 200. Moreover, the secondary pump 216 can help remove prior reagents from the automated chemical measurement system 200.

Each of the first solenoid valve 212a and second solenoid valve 212b described in FIG. 2 may be provided in the form of direct-acting solenoid valves, indirect-acting solenoid valves, normally closed or normally open solenoid valves, and/or any combination of the above. Further, one or more components of the automated chemical measurement system 200 can be communicatively coupled to the central controller 140 of FIG. 1. In one embodiment, the first solenoid valve 212a and the second solenoid valve 212b can be provided in the form of automated valves that can be controlled (i.e., opened and closed) by the central controller 140. In one embodiment, the secondary pump 216 can be communicatively coupled to the central controller 140, which can control the operation of the secondary pump 216.

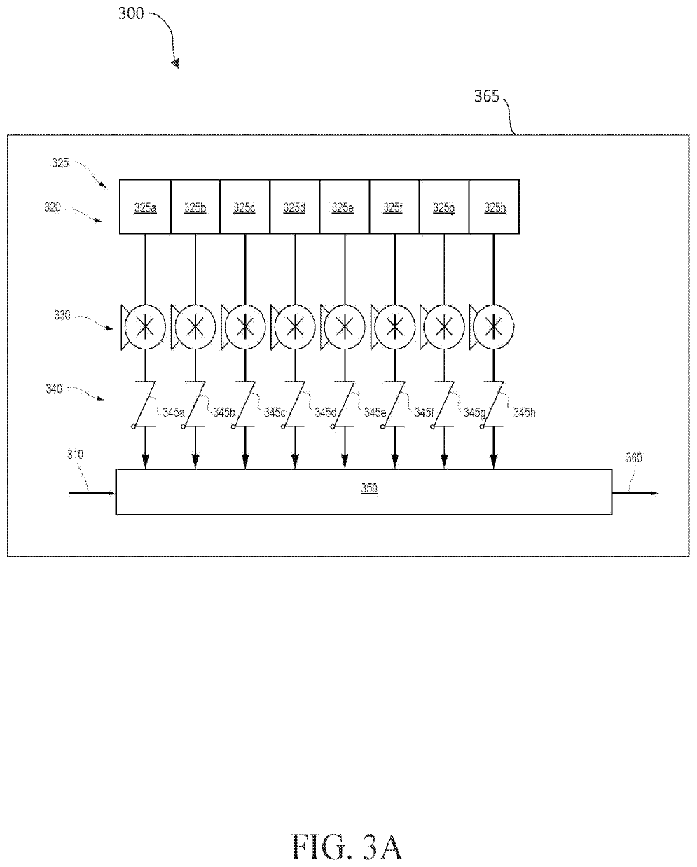

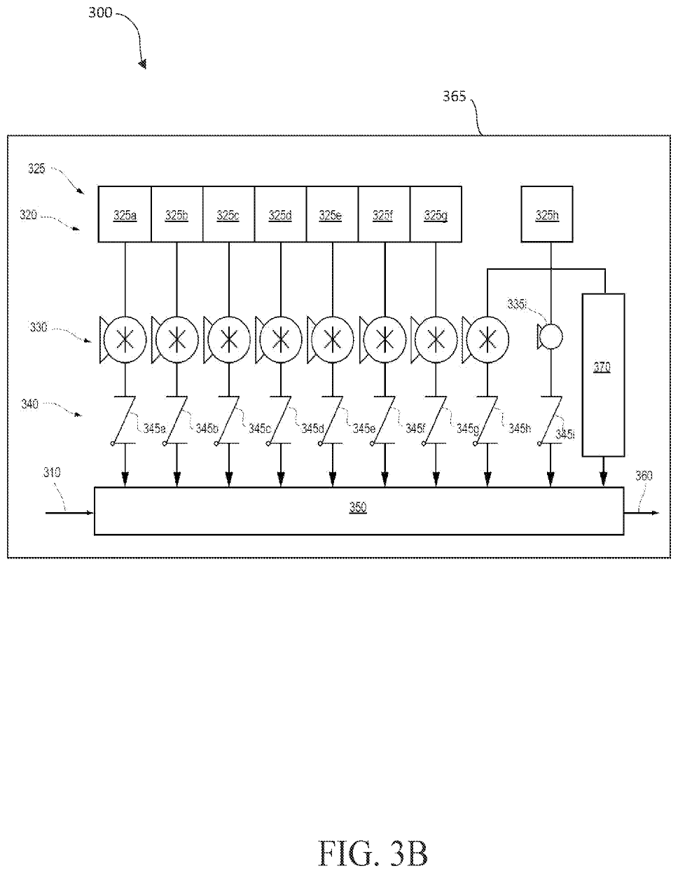

Now turning to FIG. 3A, a detailed schematic of one embodiment of a chemical reagent system 300 is shown. In some aspects, the chemical reagent system 300 is the chemical reagent system 206 of FIG. 2.

The chemical reagent system 300 can include an inlet conduit 310, a reagent cartridge bank 320, a plurality of dosage pumps 330, a plurality of check valves 340, an injection manifold 350, and an outlet conduit 360. In one embodiment, the inlet conduit 310 can be the feed conduit 202 of FIG. 2. In one embodiment, the outlet conduit 360 can be the branch conduit 204 of FIG. 2 and is in fluid communication with the automated colorimeter 208 of FIG. 2. The plurality of check valves 340 can be provided in the form of one or more of a Luer lock check valve, a swing check valve, a piston check valve, a tilting disc check valve, a diaphragm check valve, a globe valve, a septum valve, a rotary valve, a butterfly check valve, or the like.

The reagent cartridge bank 320 can comprise a plurality of reagent cartridges 325 for containing one or more chemical reagents. The chemical reagents can be chemical compounds designed to assist in analyzing one or more water quality parameters. The chemical reagents can be provided in the form of an acid digestion solution for analyzing nitrogen, an alum solution for analyzing dissolved oxygen, an aluminum chloride or barium chloride solution for analyzing sulfide, an ammonium chloride solution for analyzing biochemical oxygen demand (BOD), ammonium hydroxide for analyzing lead and/or copper, a barium diphenylaminesulfonat solution for analyzing residual chlorine, a borax solution for analyzing silica, a boric acid solution for analyzing nitrogen, a bromocresol green-methyl red indicator for analyzing alkalinity, a calcium standard solution for analyzing hardness, an iodine solution for analyzing free chlorine, a chlorine standard solution for analyzing chlorine, and any other known reagent in the art.

In one embodiment, the chemical reagent system 300 can include an insulated enclosure 365 that includes one or more reagent cartridges and allows for thermal management of the chemical reagents stored in the one or more reagent cartridges. For example, the insulated enclosure 365 can provide thermal stabilization through one or more cooling or thermal control mechanisms such as a heat sink, cooling water, a fan or other air movement device, Peltier cooling, or any other mechanical and electrical mechanisms known in the art. In one embodiment, the reagent cartridge bank 320 can be designed to provide stabilization against thermal degradation by deoxygenation or dehydration of a solvent included in the reagent cartridge bank 320. In other embodiments, chemical mechanisms known in the art can be used to stabilize the one or more reagents contained in the reagent cartridge bank 320.

With specific reference to the reagent cartridges, the reagent cartridge bank 320 may comprise a plurality of reagent cartridges 325. As shown, the reagent cartridge bank 320 includes eight reagent cartridges 325a-325h. Depending on the embodiment, the reagent cartridge bank 320 can include more or fewer reagent cartridges 325. The plurality of reagent cartridges 325 can include one or more chemical reagents. For example, each reagent cartridge of the plurality of reagent cartridges 325 can contain the same or different chemical reagents. Each reagent cartridge of the plurality of reagent cartridges 325 is in fluid communication with one or more dosage pumps of the plurality of dosage pumps 330.

In one embodiment, the chemical reagent system 300 includes the same number of reagent cartridges 325 and dosage pumps 330. As shown, the chemical reagent system 300 includes eight dosage pumps 335a-335h, with each one connected to one reagent cartridge of the plurality of reagent cartridges 325. The plurality of dosage pumps 330 can provide the driving force to deliver the one or more chemical reagents stored in the reagent cartridge bank 320 to the injection manifold 350. The plurality of dosage pumps 330 can be communicatively coupled to the central controller 140 of FIG. 1. Thus, the central controller 140 is designed to control each pump of the plurality of dosage pumps 330. Alternatively, or in addition to, the chemical reagent system 300 may include its own local controller and/or is in communication with another controller in the aquatic system 100.

The chemical reagent system 300 can include a plurality of check valves 340. The plurality of check valves 340 can be positioned downstream of the plurality of dosage pumps 330 and upstream of the injection manifold 350. As illustrated, the chemical reagent system 300 can include eight check valves 345a-345h. However, the chemical reagent system 300 can include more or fewer check valves. The plurality of check valves 340 can be designed to block the flow of the chemical reagents from entering the injection manifold 350 or designed to prevent backflow through the injection manifold 350. The plurality of check valves 340 can be communicatively coupled to the central controller 140 of FIG. 1. Thus, the central controller 140 can be configured to control each check valve of the plurality of check valves 340. In use, a first check valve 345a can be configured in an open position permitting a chemical reagent from a first reagent cartridge 325a to enter the injection manifold 350. Conversely, a second check valve 345b can be configured in a closed position, blocking a chemical reagent from a second reagent cartridge 325b from entering the injection manifold 350. In this way, the check valves 340 may be selectively configured to be in the open or closed position to distribute the associated reagent into the injection manifold 350 and into the sample of water flowing through the chemical reagent system 300.

FIG. 3B illustrates another embodiment of a chemical reagent system 302. The chemical reagent system 302 is similar to the chemical reagent system 300 of FIG. 3A, however, the chemical reagent system 302 can include an additional dosage pump 335i, check valve 325i, and a manually actuated syringe system 370.

As shown, the eighth reagent cartridge 325h, is in fluid communication with two dosage pumps 335h and 335i. The dosage pumps 335h and 335i can be the same size or different sizes. By connecting the eighth reagent cartridge 325h to two dosage pumps, the chemical reagent system 302 can have more precise control over the dosage rate of the chemical reagent delivered from the eighth reagent cartridge 325h to the injection manifold 350. An additional check valve 325i may also be provided in line with the dosage pump 335i.

Referring to both FIGS. 3A and 3B, during operation of the chemical reagent systems 300 and 302, respectively, the plurality of dosage pumps 330 and the plurality of check valves 340 can be automatically controlled by the central controller 140. In some embodiments, the central controller can be designed to adjust the dosage of the chemical regent delivered from each of the reagent cartridges 325 by adjusting one or more of the plurality of dosage pumps 330.

However, a user may desire to have an option for manual control. Therefore, as shown in FIG. 3B, the chemical reagent system 302 can include a manually actuated syringe system 370 for manually injecting one or more chemical reagents into the injection manifold 350. In this instance, the injection manifold 350 includes an associated port designed to accommodate the head/tip of the manually actuated syringe system 370.

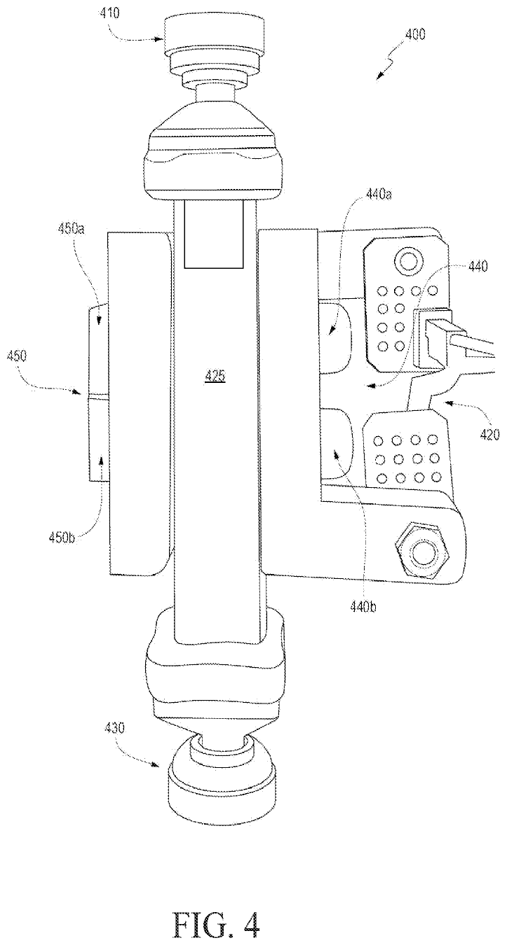

Now referring to FIG. 4, a detailed view of one embodiment of an automated colorimeter 400 is shown. In one embodiment, the automated colorimeter 400 is the automated colorimeter 208 of FIG. 2.

The automated colorimeter 400 can be provided in the form of a photodetector, spectrometer, or the like. The automated colorimeter 400 includes an inlet 410, a cuvette system 420, and an outlet 430. A pool water sample can flow into the cuvette system 420 through the inlet 410 and flow out of the cuvette system 420 through the outlet 430.

The cuvette system 420 can include a vial 425, one or more light sources 440, and one or more light detectors 450. As shown, the cuvette system 420 includes a first light source 440a, a second light source 440b, a first light detector 450a, and a second light detector 450b. However, depending on the embodiment, the cuvette system 420 can include more or fewer light sources 440 and light detectors 450. In one embodiment, the one or more light sources 440 are provided in the form of a light-emitting-diode (LED) of selected color and wavelength. In one embodiment, the one or more light sources 440 are provided in the form of a white LED. Further, each of the one or more light sources 440 can be different types and/or colors of lights. The one or more light sources 440 can be used to analyze the developed reagent chemistry of the sample water after one or more chemical reagents have been injected into a loop of a chemical measurement system, such as the automated chemical measurement system 200 of FIG. 2.

The vial 425 can be comprised of an at least partially transparent material that permits at least some light to pass through the vial 425 with little to no interference, such as Pyrex® glass, an ultra-violet (UV) Quartz, an infrared (IR) Quartz, a Sapphire, or an optically clear polymer such as polystyrene, acrylic, or polycarbonate. The cuvette system 420 can be designed to shine a light from the one or more light sources 440 through the vial 425, which holds a water sample. The one or more light detectors 450 can detect the intensity and/or color of the light that passes through the sample in the vial 425 (i.e., the one or more light detectors 450 can be designed to collect and analyze an absorbance spectrum). In this way, light is directed onto a first side of the vial 425, passes through the vial 425, and is emitted through the second side of the vial 425. It is appreciated that use of the word “side” is not to be limiting, but rather is used to illustrate that light is passed through the vial 425 such that it passes through surfaces that are provided opposite each other. The amount of light detected by the light detectors 450 can be used to determine one or more water quality parameters based on the chemical reagent injected into the water sample.

Still referring to FIG. 4, during the operation of the automated colorimeter 400, one or more chemical reagents can be combined with the water sample during the measurement of one or more water quality parameters. Further, the automated colorimeter 400 can be designed to analyze reagent degradation by comparing the results of a first measurement taken at a first time period and a second measurement taken at a second time period. For example, when performing a Total Chlorine test, the addition of an iodide-reducing agent and subsequent formation of a colored iodine solution in the mixed water sample can indicate the presence of chlorine and can be used to distinguish between a true zero free chlorine reading or a false zero free chlorine reading due to bleaching of the reagent. In another example, an ORP measurement can be used to distinguish between a true zero free chlorine reading and a false zero free chlorine reading due to bleaching of the reagent.

When the analysis of the one or more water quality parameters is complete, the water sample can be discharged from the cuvette system 420 via the outlet 430. The outlet 430 can be connected to a loop or bypass, such as the bypass conduit 112 of FIG. 1, the branch conduit 204 of FIG. 2, and/or a waste container. Further, the automated colorimeter 400 can be designed to flush the cuvette system 420 with additional water to prepare the cuvette system 420 for subsequent tests.

One or more components of the automated colorimeter 400 can be communicatively coupled to the central controller 140 of FIG. 1. In one embodiment, the one or more light sources 440 can be controlled by the central controller 140. In one embodiment, the central controller 140 can be designed to receive data from the one or more light detectors 450. Further, the central controller 140 can be designed to interpret the received data. In some embodiments, the central controller 140 can be designed to control the one or more components of the aquatic system 100 based on the interpreted data.

For example, in one non-limiting embodiment, the central controller 140 can be designed to determine one or more water quality parameters are not in compliance with a predetermined threshold or value based on the interpreted data. Further, the central controller 140 can be designed to adjust one or more components of the aquatic system 100 in response to the one or more water quality parameters being out of compliance. In one instance, the central controller 140 can determine a chlorine value is out of compliance. The central controller 140 can be designed to automatically adjust one or more of the sanitizer 124 and/or the water chemistry regulator 126. Alternatively, or in addition to, the central controller 140 can be designed to alter a user to a state of the one or more water quality parameters (i.e., whether the one or more water quality parameters are in compliance with a predetermined threshold or value). The central controller 140 can alert the user by sending a notification to the user device 150. Further, in some embodiments, the user can access the received and/or interpreted data on the user device 150.

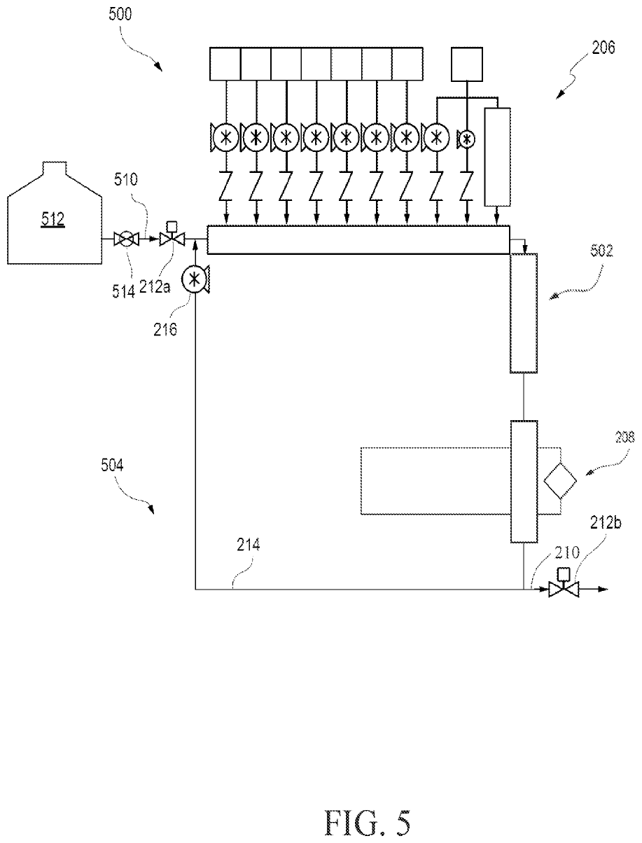

FIG. 5 illustrates a schematic of another embodiment of an automated chemical measurement system 500. The automated chemical measurement system 500 is similar to the automated chemical measurement system 200 of FIG. 2; however, a segment 504 of the automated chemical measurement system 500 can include a complementary metal-oxide-semiconductor (CMOS) sensor 502.

The CMOS sensor 502 can be positioned downstream of the chemical reagent system 206 and upstream of the automated colorimeter 208. The CMOS sensor 502 can be used to create images in digital cameras, digital video cameras, and digital CCTV cameras. The CMOS sensor 502 can include a photodiode and a CMOS transistor switch for each pixel, allowing the pixel signals to be amplified individually. The CMOS sensor 502 can be designed to analyze the impedance spectroscopy of the water sample. Thus, the CMOS sensor 502 can be designed to detect water quality parameters such as turbidity, pathogens such as bacteria and viruses, and other similar contaminates that can be identified visually.

An inlet conduit 510 of the automated chemical measurement system 500 can be in fluid communication with a water system such as a pre-mixed test water tank 512. In one embodiment, the tank 512 can be an agricultural tank or water source. A ball valve 514 can be positioned downstream of the tank 512 and upstream of the first solenoid valve 212a. In one embodiment, the outlet conduit 210 of the automated chemical measurement system 500 is in fluid communication with the tank 512. In one embodiment, the outlet conduit 210 is in fluid communication with a waste container (not shown).

It is to be understood that although FIGS. 2 and 5 show systems with an automated colorimeter 208 and/or a CMOS sensor 502, the automated chemical measurement systems described herein can also include additional water quality detection devices such as pH and ORP probes.

It is to be further understood that although FIG. 5 shows the automated chemical measurement system 500 having a chemical reagent system 206 that resembles the chemical reagent system 302 of FIG. 3B, this is not to be considered limiting. The automated chemical measurement system 500 can include any combination of the chemical reagent systems disclosed herein.

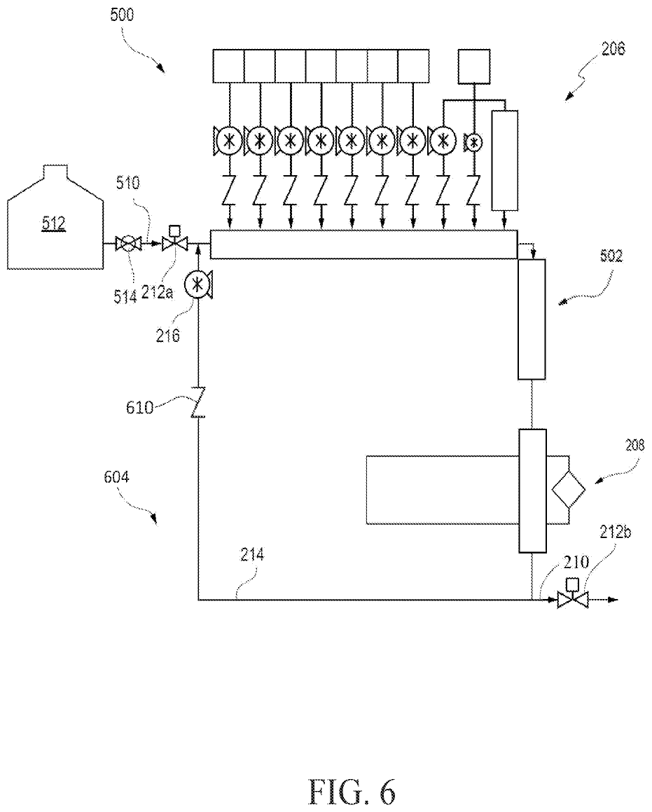

FIG. 6 illustrates a schematic of yet another embodiment of an automated chemical measurement system 600. The automated chemical measurement system 600 is similar to the automated chemical measurement system 500 of FIG. 5; however, a section 604 of the automated chemical measurement system 600 can include a check valve 610.

As shown, the check valve 610 can be positioned in the section 604 downstream of the automated colorimeter 208 and upstream of the secondary pump 216. The check valve 610 can prevent unintended backflow into the section 604.

It is to be understood that although FIG. 6 shows the automated chemical measurement system 600 having a chemical reagent system 206 that resembles the chemical reagent system 302 of FIG. 3B, this is not to be considered limiting. The automated chemical measurement system 500 can include any combination of the chemical reagent systems disclosed herein.

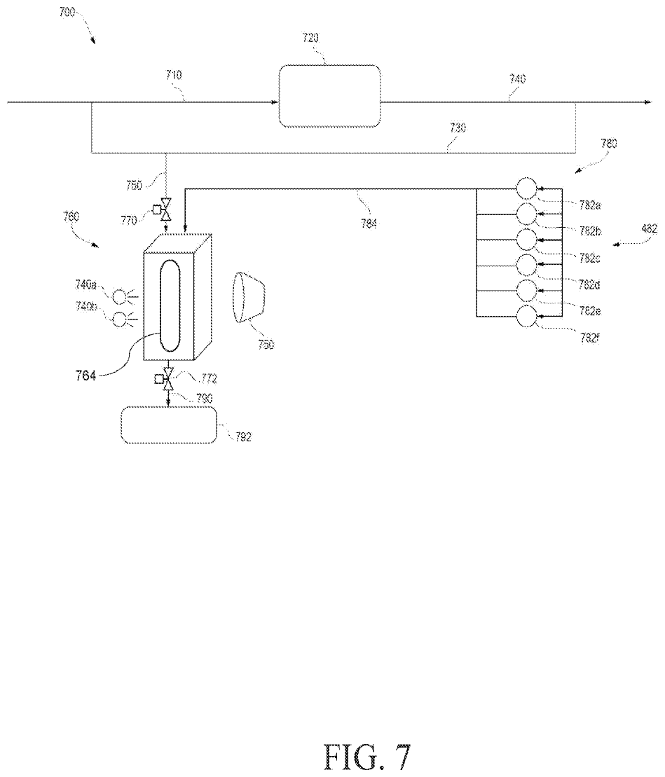

FIG. 7 illustrates a schematic of yet another embodiment of an automated chemical measurement system 700. The automated chemical measurement system 700 may be connected to an aquatic system 100 is such as the swimming pool 104 of FIG. 1.

As shown, the automated chemical measurement system 700 can include an inlet conduit 710, a pool filter 720, an outlet conduit 740, and a bypass conduit 730. In one embodiment, the inlet conduit 710 is the pump outlet conduit 110 of FIG. 1. In one embodiment, the pool filter 720 is the pool filter 114 of FIG. 1, and the outlet conduit 740 can be the same as the discharge conduit 130 of FIG. 1.

The bypass conduit 730 can permit at least a portion of the water flowing through the inlet conduit 710 to flow through a take-off conduit 735 to an automated colorimeter 760. In one embodiment, the automated colorimeter 760 is the automated colorimeter 400 of FIG. 4. The take- off conduit 735 can include a first solenoid valve 770 positioned upstream of the automated colorimeter 760 that is designed to control the flow of the water into the automated colorimeter 760.

The automated chemical measurement system 700 can further include a chemical reagent system 780. In one embodiment, the chemical reagent system 780 is the chemical reagent system 300 of FIG. 3A. In one embodiment, the chemical reagent system 780 is the chemical reagent system 302 of FIG. 3B. It is to be understood that although some components of the chemical reagent system 300 are not shown in the chemical reagent system 780, this is not to be considered limiting. Depending on the embodiment, the chemical reagent system 780 can comprise more or fewer components.

The chemical reagent system 780 can include a plurality of reagent cartridges 782. As shown, the chemical reagent system 780 includes six reagent cartridges 782a-782f. The plurality of reagent cartridges 782 can include one or more chemical reagents stored in each of the reagent cartridges of the plurality of reagent cartridges 782. Thus, similar to the chemical reagent system 300 of FIG. 3, in one embodiment, the six reagent cartridges 782a-782f can each include the same chemical reagent. Alternatively, in one embodiment, one or more reagent cartridges of the plurality of reagent cartridges 782 can include different chemical reagents. The plurality of reagent cartridges 782 can be in fluid communication with a supply conduit 784. The supply conduit 784 can supply the one or more chemical reagents contained in the plurality of reagent cartridges 782 to the automated colorimeter 760.

The automated colorimeter 760 can be designed to analyze one or more water quality parameters. The automated colorimeter 760 can be designed to automatically fill a vial 764 with a water sample from the take-off conduit 735, inject the desired chemical reagent from the chemical reagent system 780 into the vial 764 via the supply conduit 784, mix the water sample with the chemical reagent in the vial 764, analyze the water quality (via light sources 740a, 740b and a light detector 750 in a similar manner as discussed with respect to FIG. 4), empty the vial 764 through a waste conduit 790, and rinse the vial 764. The vial 764 can be rinsed with fresh water or with an untreated water sample (i.e., water from the take-off conduit 735). The water from the rinse can be directed through the waste conduit 790. In one embodiment, the waste conduit 790 can be connected to a waste container 792. The waste conduit 790 can include a second solenoid valve 772 upstream of the waste container 792 and be designed to control the water flow through the waste conduit 790 to the waste container 792. In one embodiment, the waste conduit 790 can tie into the outlet conduit 740.

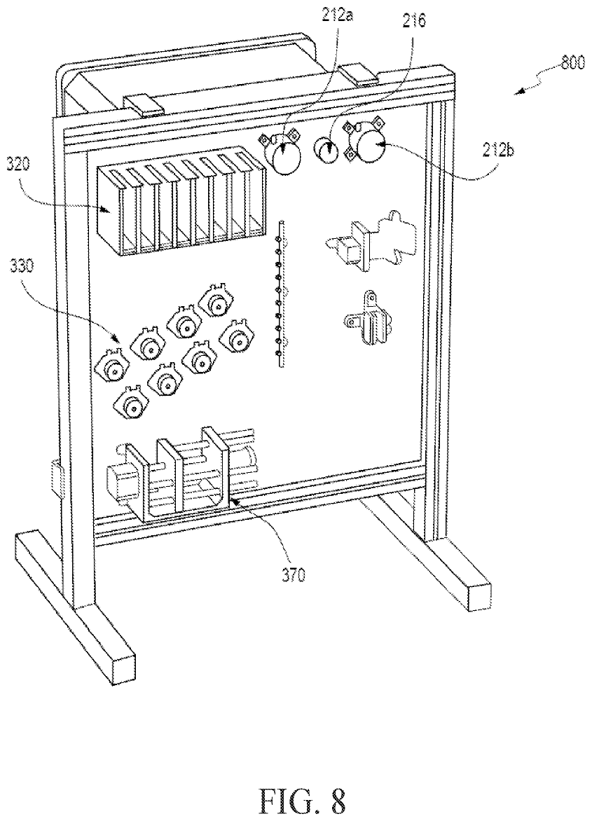

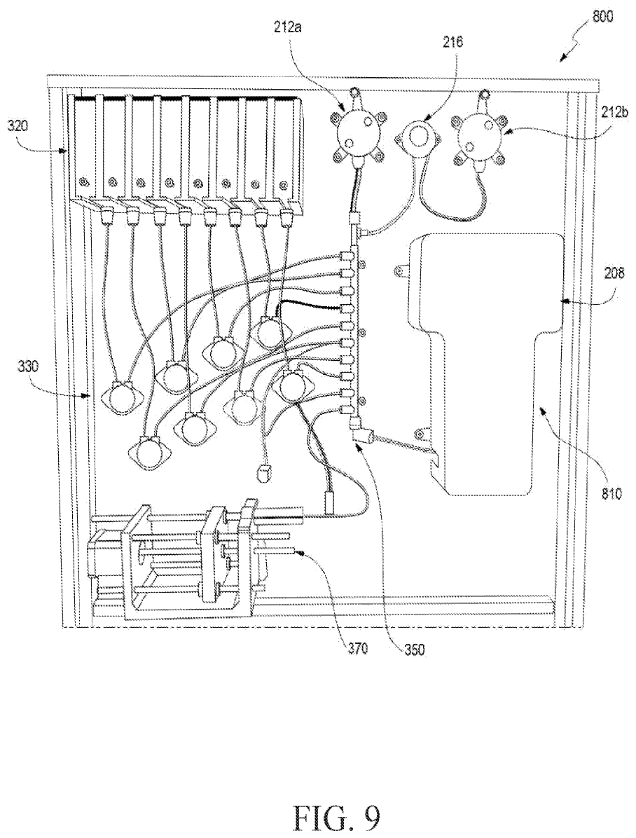

Now turning to FIGS. 8 and 9, various views of a test stand 800 for retaining one or more components of the automated chemical measurement system 118 are shown. The test stand 800 is designed to support and retain one or more components associated with the automated chemical measurement system 118 described herein.

For example, with reference to FIG. 2, the test stand 800 can include a first solenoid valve, such as the first solenoid valve 212a, and a second solenoid valve, such as the second solenoid valve 212b. Still referring to FIG. 2, the test stand 800 can include an automated colorimeter, such as the automated colorimeter 208. In some instances, the automated colorimeter may be retained in a housing 810, as shown in FIG. 9.

Further, as shown in FIGS. 8 and 9, the test stand 800 can include a chemical reagent system including one or more of the components discussed herein. As shown, the test stand includes the reagent cartridge bank 320, and the plurality of dosage pumps 330 of FIG. 3A.

A benefit of the test stand 800 is that the test stand 800 can provide an easily accessible system for retaining and/or supporting one or more components of an automated chemical measurement system. For example, a user may be able to visually inspect the components. Further, it may be easier for a user to replace or perform maintenance on one or more components because the components may be spaced far enough apart to allow easy access to each component.

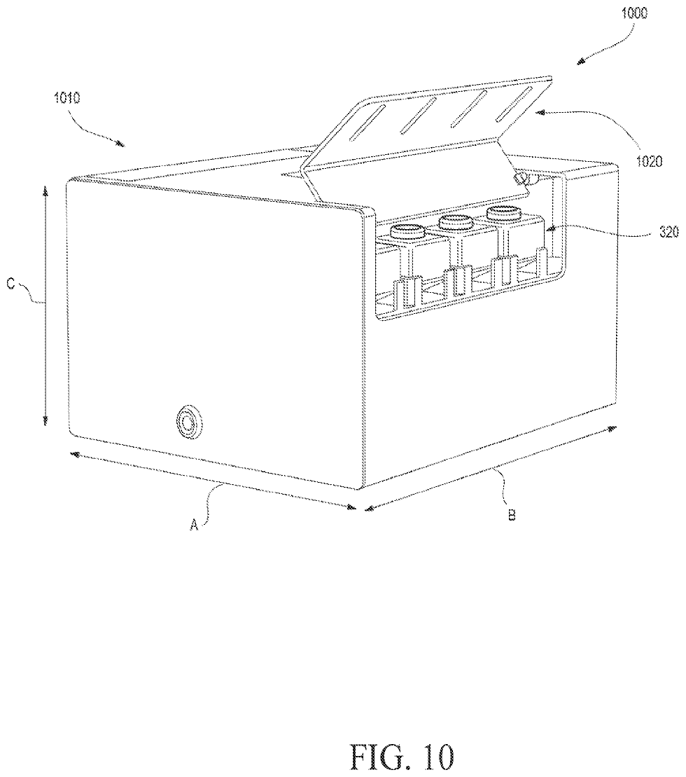

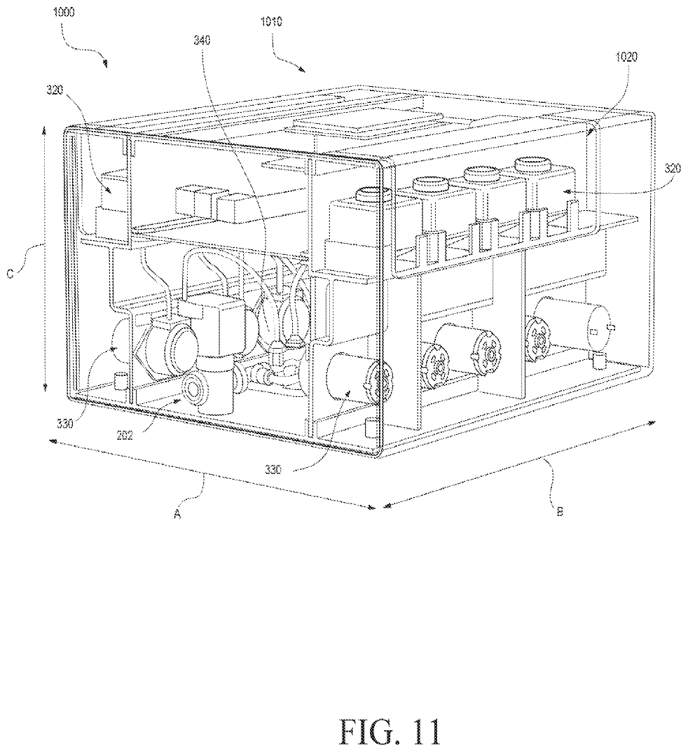

Now referring to FIGS. 10 and 11, a compact housing 1000 for retaining one or more components of the automated chemical measurement system 118, according to the embodiments described above, is shown.

The compact housing 1000 provides a similar function to the test stand 800. However, the compact housing 1000 can retain and support components of the automated chemical measurement system 118 in a more compact design as compared to the test stand 800. The compact housing 1000 can provide more protection for the components because the compact system can include a cover 1010 that surrounds one or more components. In one embodiment, the compact housing 1000 can include a selectively openable door 1020 that allows a user to access one or more components easily.

Further, the compact housing 1000 may be better suited for smaller applications where there is a limited area for storing the automated chemical measurement system. The compact housing 1000 can have a width dimension A of about 20 centimeters (cm) to about 35 cm, a length dimension B of about 25 cm to about 40 cm, and a height dimension C of about 10 cm to about 30 cm. For example, in one embodiment, the compact housing 1000 can have a width dimension A of about 26 centimeters (cm) to about 28 cm, a length dimension B of about 31 cm to about 36 cm, and a height dimension C of about 17 cm to about 21 cm.

It is to be understood that although FIGS. 8-11 may not show all the components of one or more of the automated chemical measurement systems as described above. This is not to be considered limiting. The test stand 800 and the compact housing 1000 can include more or fewer components depending on the embodiment. Further, not all components of the test stand 800, and the compact housing 1000 may be shown.

As is evident from the foregoing description, certain aspects of the present invention are not limited by the particular details of the examples illustrated herein, and it is therefore contemplated that other modifications, applications, variations, or equivalents thereof will occur to those skilled in the art. Many such changes, modifications, variations, and other uses and applications of the present constructions will, however, become apparent to those skilled in the art after considering the specification and the accompanying drawings. In addition, unless mention was made above to the contrary, it should be noted that all of the accompanying drawings are not to scale. All such changes, modifications, variations, and other uses in applications which do not depart from the spirit and scope of the present inventions are deemed to be covered by the inventions, which are limited only by the claims which follow.

What is claimed is:

1. An automated chemical measurement system for testing a sample of water from a swimming pool, comprising:
a conduit that provides a sample of swimming pool water to the automated chemical measurement system;
a chemical reagent system that injects one or more chemical reagents into the sample of swimming pool water; and
a colorimeter downstream and in fluid communication with the chemical reagent system, the colorimeter designed to analyze the sample of swimming pool water.

2. The automated chemical measurement system of claim 1, wherein the chemical reagent system includes a reagent cartridge bank having the one or more chemical reagents, at least one pump, at least one valve to selectively isolate the one or more chemical reagents from the sample of swimming pool water, and an injection manifold.

3. The automated chemical measurement system of claim 1 further including a first valve positioned in the conduit upstream of the chemical reagent system for controlling a flow of the sample of swimming pool water into the chemical reagent system of the automated chemical measurement system, and a second valve positioned in the conduit downstream of the colorimeter designed for controlling the flow of the sample of swimming pool water out of the automated chemical measurement system.

4. The automated chemical measurement system of claim 2, wherein the reagent cartridge bank comprises a chemical mechanism for thermally stabilizing the one or more chemical reagents provided in the form of deoxygenation of the one or more chemical reagents and a solvent, or dehydration of the one or more chemical reagents and the solvent.

5. The automated chemical measurement system of claim 2, wherein the reagent cartridge bank comprises a mechanical mechanism or an electrical mechanism for thermally stabilizing the one or more chemical reagents provided in the form of one or more of a heat sink, a flow of cooling water provided to the reagent cartridge bank, a fan or other air movement device, a Peltier cooling system, or a refrigeration cycle.

6. The automated chemical measurement system of claim 1, wherein the colorimeter further includes a pH or an ORP probe.

7. The automated chemical measurement system of claim 6, wherein the ORP probe differentiates between a true zero free chlorine reading or a false zero free chlorine reading.

8. The automated chemical measurement system of claim 1, wherein the colorimeter is provided in the form of a photodetector, spectrometer, or photometric analyzer.

9. The automated chemical measurement system of claim 8, wherein the colorimeter collects and analyzes an absorbance spectrum to ensure the one or more chemical reagents is functioning as anticipated.

10. The automated chemical measurement system of claim 9, wherein the colorimeter is designed to run a chemical measurement test on the sample of swimming pool water by comparing a result of a first measurement taken at a first time period and a second measurement taken at a second time period.

11. The automated chemical measurement system of claim 10, wherein the colorimeter provides information on reagent degradation by comparing the first measurement and the second measurement.

12. A system for analyzing water quality of a water sample of an aquatic system, comprising:
an inlet designed to deliver the water sample;
a chemical reagent system downstream of the inlet, wherein the chemical reagent system is designed to inject one or more reagents into the water sample;
a colorimeter downstream of the chemical reagent system, the colorimeter comprising:
a vial designed to contain the water sample;
at least one light source designed to emit light towards a first side of the vial; and
at least one light detector designed to detect the emitted light on a second side of the vial; and
a controller that adjusts a dosage of the one or more reagents injected into the water sample, receives data from the at least one light detector, and analyzes the water quality of the water sample based on the received data.

13. The system for analyzing water quality of a water sample of claim 12, wherein the data includes how much emitted light was detected by the at least one light detector.

14. The system for analyzing water quality of a water sample of claim 12, wherein the chemical reagent system further includes a manually actuated syringe designed to permit a user to manually inject a reagent into the water sample.

15. The system for analyzing water quality of a water sample of claim 12, wherein the system is located in a bypass loop of the aquatic system.

16. The system for analyzing water quality of a water sample of claim 12, wherein the aquatic system includes a sanitizer, a water chemistry regulator, a filter, and a heater, and wherein the controller controls at least one of the sanitizer, the water chemistry regulator, the filter, and the heater in response to the analyzed water quality.

17. The system for analyzing water quality of a water sample of claim 12, wherein the chemical reagent system comprises;
a reagent cartridge bank designed to contain one or more chemical reagents;
at least one pump downstream of the reagent cartridge bank, the at least one pump designed to provide a driving force to deliver the one or more chemical reagents to the water sample;
at least one valve downstream of the at least one pump, the at least one valve designed to control a flow of the one or more chemical reagents; and
an injection manifold downstream of the at least one valve, wherein the injection manifold is designed to inject the one or more chemical reagents into the water sample.

18. The system for analyzing water quality of a water sample of claim 17, wherein the controller is configured to control a flow rate of the one or more chemical reagents by adjusting the at least one valve from a first closed position to a second open position.

19. The system for analyzing water quality of a water sample of claim 12 further including a complementary metal-oxide-semiconductor sensor.

20. A method of analyzing one or more water quality parameters of a water sample in a closed loop aquatic system, comprising:
delivering the water sample to a vial;
providing a reagent test bank having a reagent cartridge that contains a chemical reagent, a pump designed to control a dosage of the chemical reagent, a valve designed to control a flow of the chemical reagent, and an injection manifold designed to inject the chemical reagent into the water sample;
adding the chemical reagent to the water sample;
emitting a light from a light source toward the vial;
detecting the emitted light with a light detector adjacent the vial; and
determining the one or more water quality parameters based on the detected emitted light.
